(19) United States
(12) Patent Application Publication
(10) Pub. No.: US 2024/0255478 A1
(43) Pub. Date: Aug. 1, 2024

(54) DATA PROCESSING DEVICE FOR METABOLITE ANALYSIS

(71) Applicant: SHIMADZU CORPORATION, Kyoto-shi (JP)

(72) Inventor: Yuki NAKAGAWA, Kyoto-shi (JP)

(73) Assignee: SHIMADZU CORPORATION, Kyoto-shi (JP)

(21) Appl. No.: 18/481,469

(22) Filed: Oct. 5, 2023

(30) Foreign Application Priority Data
Jan. 31, 2023 (JP) 2023-013492

(51) Int. Cl. G01N 30/88; G01N 30/72; G16B 40/10; G16B 45/00

(52) U.S. Cl. CPC G01N 30/88; G01N 30/7206; G01N 30/7233; G16B 40/10; G16B 45/00; G01N 2030/025; G01N 2030/027; G01N 2030/8813

(57) ABSTRACT

In a data processing device for metabolite analysis, a metabolite-measurement-result storage section holds a metabolite identified from analysis data acquired by analyzing a test sample with one or more analyzers and a quantitative measurement result of the metabolite. A metabolism-map-data storage section holds metabolism-map display data for one or more metabolism maps each of which shows metabolites along with respective quantitative measurement results, with each metabolite having a showing area to which analyzer information representing the type of analyzer capable of detecting the metabolite is to be added. A display processor retrieves a quantitative measurement result for each metabolite on a predetermined metabolism map from the metabolite-measurement-result storage section and displays the quantitative measurement result by inserting it into the showing area for the metabolite on the metabolism map, when the metabolism-map display data stored in the metabolism-map-data storage section is read to display the predetermined metabolism map.

### TECHNICAL FIELD

The present invention relates to a data processing device for metabolite analysis configured to create a metabolism map based on analysis data of a test sample.

### BACKGROUND ART

Metabolite analysis employing a gas chromatograph mass spectrometer (GC/MS) or liquid chromatograph mass spectrometer (LC/MS) has been commonly performed for the diagnosis and treatment of diseases accompanied with metabolic abnormalities, as well as for the research and development (or the like) of therapeutic agents for those diseases. Metabolite analysis employing a GC/MS or LC/MS has also been performed for other purposes, such as a study of the safety of pharmaceuticals, health foods or drugs (or the like), a pharmacological study on the toxicity of those products, as well as a metabolome analysis of flora and fauna.

In a metabolite analysis, a chart called a “metabolism map” is popularly used, which shows metabolic pathways within the body of a human or other kinds of living organisms (Non Patent Literature 1). A metabolic map shows various compounds (metabolites) produced in the course of metabolism, as well as chemical reactions, enzymes involved in the metabolism, and other related elements, allowing the metabolic sequence to be easily understood visually. With such a metabolism map, it is possible to easily determine what kinds of metabolites are present in a test sample, such as a urine sample, taken from a human or other kinds of living organisms.

For example, in the case of determining whether or not various metabolites included in a given metabolism map are contained in a test sample, a GC/MS and/or LC/MS is used to analyze metabolites contained in the test sample, and each of the metabolites contained in the test sample is identified from a mass spectrum or mass chromatogram corresponding to a chromatogram peak obtained from the result of the analysis. For each metabolite, a quantitative value of the metabolite, or a bar chart representing the quantitative value, is inserted into the metabolism map as a measurement result related to that metabolite. The tasks described thus far are repeated for all metabolites included in the metabolism map to complete a metabolism map which reflects the analysis result obtained for the test sample using the GC/MS or LC/MS.

However, the task of creating a metabolism map from an analysis result obtained for a test sample is considerably complex and burdensome for the analysis operator. Accordingly, a data processing device for metabolite analysis has been proposed which receives an input of an analysis result obtained for a test sample and automatically performs the tasks from a data analysis based on the measurement result to the creation of a metabolism map. This device has a storage section in which metabolism-map display data for displaying a metabolism map on a display screen is stored. After the analysis of the data of the analysis result obtained for the test sample has been completed, the device reads the metabolism-map display data from the storage section, creates a metabolism map in which the quantitative values (or similar data) of the metabolites are inserted, and displays the map on the display screen.

### CITATION LIST

### Non Patent Literature

Non Patent Literature 1: Zenzaburo Tozuka, et al., “Multi omics by LC-MS/MS to search small molecule ligands of nuclear receptors to control transcription of pharmaceutical active proteins for drug discovery”, Trends in Res, Vol 1(2), 2018, pp. 1-2

### SUMMARY OF INVENTION

### Technical Problem

Some of the metabolites on the metabolism map can be detected with both GC/MS and LC/MS, while others can only be detected by either GC/MS or LC/MS. There are also metabolites that can be detected with neither GC/MS nor LC/MS. Accordingly, when there is a metabolite having no measurement result inserted among the metabolites on the metabolism map, it is possible that the metabolite in question is actually contained in the test sample. However, it has been difficult to determine, from a view of the metabolism map, whether or not the metabolite concerned may possibly be contained in the test sample.

Thus, in a data processing device for metabolite analysis which creates a metabolism map that reflects an analysis result obtained for a test sample, the problem to be solved by the present invention is to allow users to easily determine, from the metabolism map, whether or not a specific metabolite may possibly be contained in the test sample.

### Solution to Problem

A data processing device for metabolite analysis according to the present invention developed for solving the previously described problem includes:
a metabolite-measurement-result storage section in which a metabolite identified based on analysis data acquired by analyzing a test sample with one or more analyzers and a quantitative measurement result of the metabolite are stored;
a metabolism-map-data storage section in which metabolism-map display data for displaying, on a display screen, a metabolism map is stored for one or more metabolism maps, with each metabolism map showing a plurality of metabolites along with respective quantitative measurement results, and each metabolite having a showing area to which analyzer information representing the type of analyzer capable of detecting the metabolite is to be added; and
a display processor configured to retrieve, for each metabolite on a predetermined metabolism map, a quantitative measurement result corresponding to the metabolite from the metabolite-measurement-result storage section and to display the quantitative measurement result by inserting the quantitative measurement result into the showing area for the metabolite on the metabolism map, when the metabolism-map display data stored in the metabolism-map-data storage section is read to display the predetermined metabolism map on the display screen.

### Advantageous Effects of Invention

In the data processing device for metabolite analysis according to the present invention, when a metabolism map reflecting an analysis result obtained for a test sample is to be displayed on the display screen, a piece of information which represents the type of analyzer capable of detecting the metabolite is added to the showing area for each metabolite included in the metabolism map. Therefore, when a metabolite having no quantitative measurement result inserted is present among the metabolites included in the metabolism map, the user can easily determine, from the analyzer information added to the showing area for that metabolite, whether or not the metabolite concerned may possibly be contained in the test sample. Specifically, when the type of each of the one or more analyzers used for analyzing the test sample is different from that of the analyzer with which that metabolite can be detected, it is possible to determine that the metabolite concerned may possibly be contained in the test sample.

### BRIEF DESCRIPTION OF DRAWINGS

FIG. 1 is a block configuration diagram of a metabolite analyzing system employing a data processing device for metabolite analysis according to the present invention.

FIG. 2 is a flowchart showing a procedure for creating a metabolism map.

FIG. 3 is a diagram showing a display example of a metabolism map before an analysis of a test sample.

FIG. 4 is a diagram showing a display example of a metabolism map after an analysis of a test sample.

FIG. 5 is a diagram showing a metabolism map after an analysis of a test sample, with the names of the derivatization products of the metabolites displayed on the map.

### DESCRIPTION OF EMBODIMENTS

One embodiment of a metabolite analyzing system employing a data processing device for metabolite analysis according to the present invention is hereinafter described with reference to the drawings.

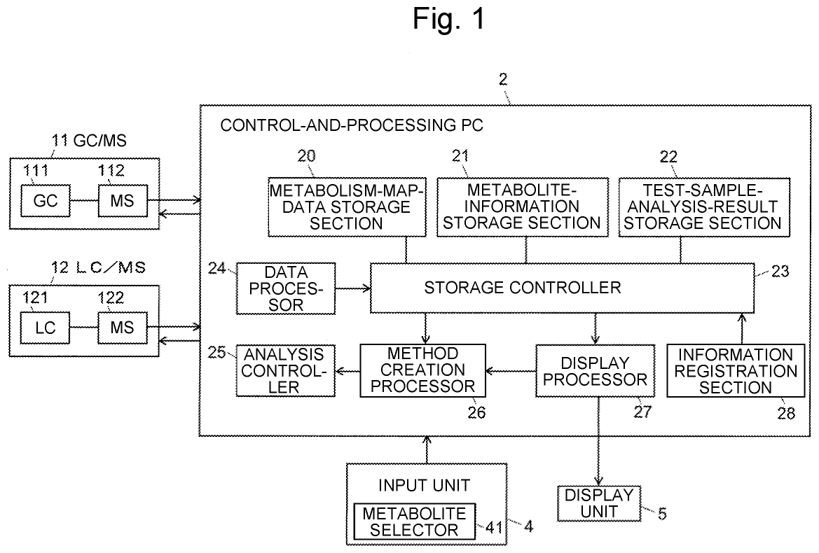

FIG. 1 is an overall configuration diagram of the metabolite analyzing system according to the present embodiment. This metabolite analyzing system includes a GC/MS 11 and an LC/MS 12 as the analyzers, as well as a control-and-processing personal computer (PC) 2. The GC/MS 11 includes a GC unit 111 and an MS unit 112, while the LC/MS 12 includes an LC unit 121 and an MS unit 122. The PC 2 is configured to control the operations of the GC/MS 11 and LC/MS 12 as well as to process analysis data collected with the GC/MS 11 and those collected with the LC/MS 12.

The control-and-processing PC 2 has an input unit 4 and a display unit 5 connected as user interfaces. The control-and-processing PC 2 is a generic personal computer on which dedicated control-and-processing software is installed as application software. Executing this software on the PC enables this computer to perform a normal control and data processing for the GC/MS 11 as well as carry out characteristic control-and-processing operations as will be described later. That is to say, the control-and-processing PC 2 corresponds to the data processing device for metabolite analysis according to the present invention.

The data processing device for metabolite analysis realized by the control-and-processing PC 2 includes, as its functional blocks, a metabolism-map-data storage section 20, metabolite-information storage section 21, test-sample-analysis-result storage section 22, storage controller 23, data processor 24, analysis controller 25, method creation processor 26, display processor 27, information registration section 28, and metabolite selector 41 which is included in the input unit 4.

The metabolism-map-data storage section 20 is configured to store data constituting one or more metabolism maps (normally, a plurality of metabolism maps). The metabolite-information storage section 21 is configured to store analysis-related information for each metabolite that can be analyzed by one or both of the GC/MS 11 and LC/MS 12, where the analysis-related information includes analysis conditions, inclusive of data processing conditions, as well as analysis results obtained for known standard samples, such as retention indices, mass spectra and characteristic ion information (e.g., mass-to-charge ratios of ions and intensity ratio of multiple ions). The analysis results obtained for standard samples (such as the mass spectra and retention indices) and included in the analysis-related information prepared for each metabolite may be obtained by actual measurements of standard samples containing the metabolites concerned by means of the GC/MS 11 and LC/MS 12 in the present system, or those pieces of information published in academic papers or similar sources may also be used. It should be noted that a measurement for one metabolite may be conducted under different analysis conditions using the GC/MS 11 or LC/MS 12. Accordingly, the number of pieces of analysis-related information related to one metabolite is not always one; it may be two or more.

The test-sample-analysis-result storage section 22 is configured to store information for each test sample which is not a standard sample and contains unknown components, where the information includes: the retention index, mass spectrum and other analysis results acquired by analyzing the test sample by the GC/MS 11 and/or LC/MS 12; the analysis conditions under which the analysis was performed; the analysis result obtained for a standard sample (e.g., mass spectrum and retention index); as well as metabolites identified from the result of the analysis of the test sample, and quantitative measurement results obtained for those metabolites. The test-sample-analysis-result storage section 22 corresponds to the metabolite-measurement-result storage section in the present invention. The storage sections 20, 21 and 22 may be provided in different storage areas in one memory device (e.g., HDD or SSD), or they may be provided in separate memory devices.

In the present embodiment, the data constituting a metabolism map stored in the metabolism-map-data storage section 20 (this data is hereinafter called the “metabolism map data”) includes metabolism-map display data for displaying, on the screen of the display unit 5, the names of a plurality of metabolites constituting the metabolism map, in such a manner that those names are arranged along the metabolic pathways, with each name accompanied by a rectangular frame showing an area into which a quantitative measurement result obtained for the metabolite is to be inserted. The metabolism-map display data includes analyzer information which represents the type of analyzer with which the metabolite concerned can be detected. The analyzer information will be described later.

For the present embodiment, it is assumed that the analysis-related information stored for each metabolite in the metabolite-information storage section 21 includes information concerning derivatization, which is one of the pretreatments in the GC/MS 11 and/or LC/MS 12. This information includes the derivatization reagent used for the derivatization, the derivatization product formed by a reaction of the metabolite with the derivatization reagent, as well as the nature of the same product.

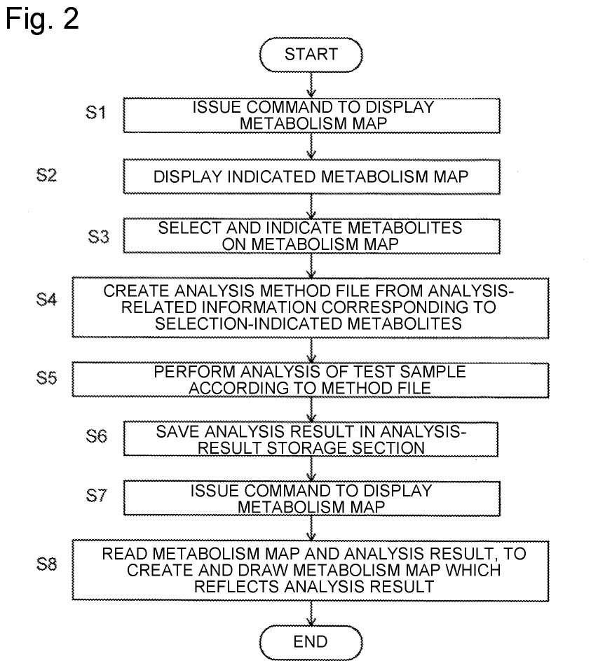

Next, one example of an analysis procedure for identifying metabolites contained in a test sample using the metabolite analyzing system having the previously described configuration is described according to the flowchart in FIG. 2.

A user (operator) initially issues a command through the input unit 4 to display a metabolism map on which a desired metabolic pathway is shown (Step 1). In response to this command, the display processor 27 reads the corresponding metabolism map data from the metabolism-map-data storage section 200 via the storage controller 23 and draws the metabolism map on the screen of the display unit 5. It also reads and acquires, from the metabolite-information storage section 21, analysis-related information corresponding to the metabolites on the metabolism map (Step 2). The display processor 27 may preferably be configured to allow the user to select the desired metabolism map, e.g., by the name of the metabolism map, when there are a plurality of metabolism maps.

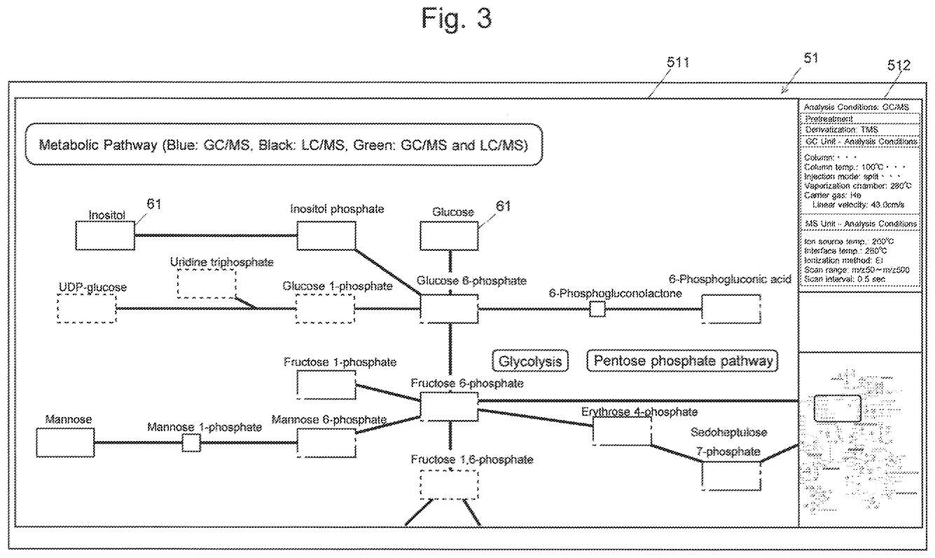

On the metabolism map shown on the screen of the display unit 5, the user selects and indicates, through the metabolite selector 41, the metabolic pathway whose presence or absence in the test sample should be determined (Step 3). FIG. 3 shows one example of the display screen shown in this situation (which is hereinafter called the “pre-analysis display screen 51”). The pre-analysis display screen 51 has a main area 511, within which a metabolism map including the metabolic pathway selected through the metabolite selector 41 (“partial metabolism map”) is shown, as well as a sub-area 512 on the right side, within which the entirety of the metabolism map (“entire metabolism map”) is shown in the lower section and details of the analysis conditions in the upper section. The portion surrounded by the rectangular frame in the entire metabolism map shown in the lower section of the sub-area 512 corresponds to the partial metabolism map shown in the main area 511.

On the metabolism map shown on the display screen, rectangular frames 61 each of which forms an area into which the quantitative measurement result obtained for a metabolite included in the metabolism map is to be inserted are arranged along the metabolic pathway, with each frame accompanied by the name of the corresponding metabolite. As shown in FIG. 3, the metabolism map displayed in the pre-analysis display screen 51 has no quantitative measurement results inserted in the rectangular frames 61 for the metabolites. With the rectangular frames 61 thus being blank, this map is also called a “blank map for the metabolism map”.

In the present embodiment, each of the rectangular frames 61 for the metabolites on the metabolism map shown on the display screen is given a color representing the type of analyzer as the analyzer information which shows whether the analyzer with which the metabolite concerned can be detected is the GC/MS 11 or LC/MS 12. Specifically, the rectangular frame 61 for a metabolite which can only be detected with the GC/MS 11 is shown in blue. The rectangular frame 61 for a metabolite which can only be detected with the LC/MS 12 is shown in black. The rectangular frame 61 for a metabolite which can be detected with both the GC/MS 11 and LC/MS 12 is shown in green. It should be noted that the rectangular frames shown in blue, black and green are represented by the solid line, dashed line, and long-dashed short-dashed line in FIG. 3, respectively.

After the partial metabolism map selection-indicated by the user has been completed, the method creation processor 26 obtains analysis-related information corresponding to the metabolites included in the selection-indicated partial metabolism map, creates a method file including various analysis parameters and data-processing parameters necessary for performing the analysis by one or both of the GC/MS 11 and LC/MS 12 (Step 4), and sends this file to the analysis controller 25. Upon receiving a command to initiate the analysis, the analysis controller 25 operates the GC/MS 11 and/or LC/MS 12 according to this method file to conduct the analysis on the test sample (Step 5).

The analysis data collected with the GC/MS 11 and/or LC/MS 12 is sent to the data processor 24. The data processor 24 performs a data processing based on the data processing conditions described in the method file. Since the method file contains the retention index, mass spectrum, characteristic ion information and other pieces of relevant information determined for each metabolite, it is possible to easily detect each metabolite by using those pieces of information. For example, since the retention time can be inferred from the retention index, a retention-time range with an appropriate amount of time allowance before and after the inferred retention time can be set, and a peak which seems to correspond to the target metabolite is extracted from this narrowed range of retention time on the chromatogram. By comparing the pattern of a mass spectrum acquired by an actual measurement around this peak with that of the mass spectrum of the standard sample and determining their similarity, it is possible to determine, with a high level of certainty, whether or not the peak corresponds to the target metabolite. The data processor 24 can also calculate a quantitative value of the metabolite based on appropriate information, such as a peak area on a mass chromatogram at a mass-to-charge ratio corresponding to the identified metabolite. Using the chromatograms along with the mass spectrum for the identification in the previously described manner makes the detection of the metabolite easier and highly reliable as compared to the case where the peak pattern of the mass spectrum is solely used for the detection.

After the metabolites contained in the test sample have been sequentially identified by the data processor 24 and their quantitative values have been calculated, these analysis results are stored in the test-sample-analysis-result storage section 22 via the storage controller 23 (Step 6). If there is a metabolite for which a plurality of derivatization products were formed through a derivatization process performed as a pretreatment before the analysis, the quantitative values of those derivatization products are related to that metabolite and stored in the test-sample-analysis-result storage section 22. The analysis result (e.g., mass spectrum and retention index) and analysis conditions for the standard sample stored in the metabolite-information storage section 21 may also be stored as they are for each test sample, or alternatively, only the correspondence relationship with the information stored in the metabolite-information storage section 21 may be recorded.

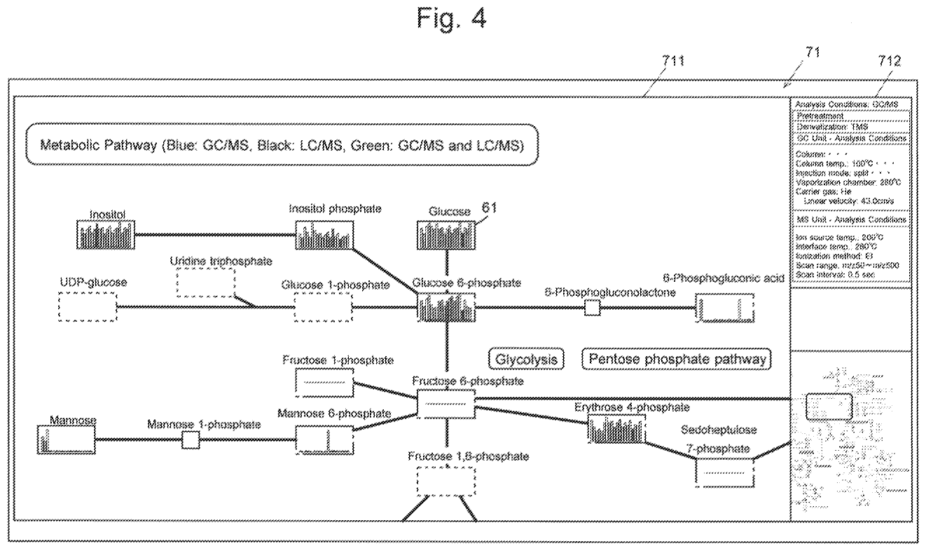

At a later time, the user issues a command from the input unit 4 to create a metabolism map (Step 7). Then, the display processor 27 reads the metabolism map data from the metabolism-map-data storage section 20 via the storage controller 23. It also reads, from the test-sample-analysis-result storage section 22, the quantitative measurement results obtained for the metabolites on the metabolism map and writes those results on the metabolism map as the analysis result obtained for the test sample related to this metabolism map. FIG. 4 is a diagram showing one example of the display screen shown in this situation (which is hereinafter called the “post-analysis display screen 71”). Similar to the pre-analysis display screen 51, the post-analysis display screen 71 has a main area 711 within which a partial metabolism map is shown, as well as a sub-area 712 within which the entire metabolism map is shown in the lower section and details of the analysis conditions in the upper section. In the example shown in FIG. 4, a bar chart of the quantitative value of the metabolite contained in a plurality of test samples is inserted into each rectangular frame 61 on the metabolism map as the quantitative measurement result obtained for the metabolite, while a horizontal bar is shown in the rectangular frames 61 for metabolites which have not been selection-indicated. The rectangular frames 61 for metabolites which have been selection-indicated yet have not been detected are left blank.

By viewing the post-analysis display screen 71 configured in this manner, the user can determine, from the display color of the rectangular frame 61, whether or not a metabolite which has been selection-indicated yet has not been detected may potentially be present in the test sample: For example, in the case where the device used for analyzing the test sample was the LC/MS 12, when there is a metabolite with the rectangular frame 61 left blank and displayed in blue (i.e., a metabolite that can only be detected with the GC/MS 11), the user can determine that the metabolite may possibly be detected by changing the analyzer to the GC/MS 11. As another example, in the case where the device used for analyzing the test sample was the LC/MS 12, when there is a metabolite with the rectangular frame 61 left blank and displayed in green (i.e., a metabolite that can be detected by both the GC/MS 11 and LC/MS 12), the user can determine that the metabolite, which could not be detected by the LC/MS 12, may possibly be detected with the GC/MS 11.

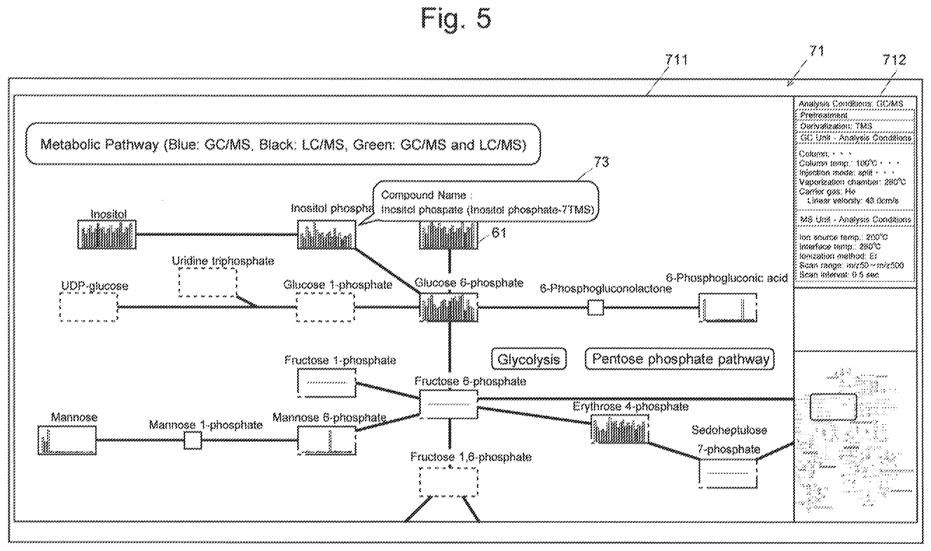

Furthermore, for a metabolite for which the quantitative values of a plurality of derivatization products are stored in the test-sample-analysis-result storage section 22, the storage controller 23 in the present embodiment reads, from the test-sample-analysis-result storage section 22, the quantitative measurement result obtained for a derivatization product which satisfies a predetermined condition, and writes it on the metabolism map. The “predetermined condition” may be the selected derivatization product being superior to the other derivatization products in detection sensitivity, in detection reproducibility, or in stability against heat or chemicals. For example, when inositol phosphate is to be detected as a metabolite, trimethylsilylation is performed as a pretreatment for the test sample. In this case, a quantitative value of a derivatization product (inositol phosphate-7TMS) formed by the pretreatment is read from the test-sample-analysis-result storage section 22 as a quantitative measurement result obtained for inositol phosphate. In the case where the quantitative measurement result shown in the rectangular frame 61 on the metabolism map is a quantitative value of a derivatization product, a pop-up window 73 showing the name of the derivatization product is displayed, as shown in FIG. 5 for example, when the rectangular frame 61 on the post-analysis display screen 71 is indicated. By viewing this pop-up window 73, the user can check which derivatization product of the metabolite has yielded the quantitative measurement result inserted in that rectangular frame 61.

The metabolite analyzing system according to the previous embodiment has one type of GC/MS 11 and one type of LC/MS 12 as the analyzers. It may have two or more devices of the same or different types for at least one of the GC/MS 11 and LC/MS 12. For example, when a plurality of GC/MSs are used at the same time, a different type of column can be connected to each GC/MS, or multiple analyses can be performed in parallel under different analysis conditions, which contributes to a reduction of the analyzing time.

The metabolite analyzing system according to the previous embodiment is configured so that each rectangular frame into which a quantitative measurement result obtained for a metabolite is to be inserted on the metabolism map is shown in a color corresponding to the type of analyzer capable of detecting the metabolite. It may also be configured so that the inner area of the rectangular frame, or the name of the metabolite, is shown in a color corresponding to the type of analyzer capable of detecting the metabolite. A mark representing the type of analyzer may also be shown in the vicinity of the rectangular frame or the name of the metabolite.

### Modes

It is evident for a person skilled in the art that the previously described illustrative embodiment is a specific example of the following modes of the present invention.

(Clause 1) A data processing device for metabolite analysis according one mode of the present invention includes:
a metabolite-measurement-result storage section in which a metabolite identified based on analysis data acquired by analyzing a test sample with one or more analyzers and a quantitative measurement result of the metabolite are stored;
a metabolism-map-data storage section in which metabolism-map display data for displaying, on a display screen, a metabolism map is stored for one or more metabolism maps, with each metabolism map showing a plurality of metabolites along with respective quantitative measurement results, and each metabolite having a showing area to which analyzer information representing the type of analyzer capable of detecting the metabolite is to be added; and
a display processor configured to retrieve, for each metabolite on a predetermined metabolism map, a quantitative measurement result corresponding to the metabolite from the metabolite-measurement-result storage section and to display the quantitative measurement result by inserting the quantitative measurement result into the showing area for the metabolite on the metabolism map, when the metabolism-map display data stored in the metabolism-map-data storage section is read to display the predetermined metabolism map on the display screen.

In the data processing device for metabolite analysis according to Clause 1, when a metabolism map reflecting an analysis result obtained for a test sample is to be displayed on the display screen, a piece of information which represents the type of analyzer capable of detecting the metabolite is added to the showing area for each metabolite included in the metabolism map. Therefore, when a metabolite having no quantitative measurement result inserted is present among the metabolites included in the metabolism map, the user can easily determine, from the analyzer information added to the showing area for that metabolite, whether or not the metabolite concerned may possibly be contained in the test sample. Specifically, when the type of the analyzer used for analyzing the test sample is different from that of the analyzer with which that metabolite can be detected, it is possible to determine that the metabolite concerned may possibly be contained in the test sample.

(Clause 2) In general, an analyzer used for metabolite analysis is a gas chromatograph mass spectrometer or liquid chromatograph mass spectrometer. Accordingly, in a data processing device for metabolite analysis according to Clause 2, which is one mode of the data processing device for metabolite analysis according to Clause 1, the one or more analyzers are a gas chromatograph mass spectrometer and a liquid chromatograph mass spectrometer.

The data processing device for metabolite analysis according to Clause 2 can create a useful metabolism map for a wide variety of metabolite analyses with various aims, such as the diagnosis and treatment of diseases accompanied with metabolic abnormalities, research and development (or the like) of therapeutic agents for those diseases, a study of the safety of pharmaceuticals, health food or drugs (or the like), a pharmacological study on the toxicity of those products, as well as a metabolome analysis of flora and fauna.

(Clause 3) In a data processing device for metabolite analysis according to Clause 3, which is one mode of the data processing device for metabolite analysis according to Clause 1 or 2, the analyzer information added to the showing area for each metabolite on the metabolism map is color information for displaying at least a portion of the showing area in a color corresponding to the type of analyzer.

In the data processing device for metabolite analysis according to Clause 3, the user can recognize the type of analyzer capable of detecting the metabolite, by visually checking the color of at least a portion of the showing area provided for each metabolite on the metabolism map.

(Clause 4) In a data processing device for metabolite analysis according to Clause 4, which is one mode of the data processing device for metabolite analysis according to one of Clauses 1-3,
the metabolite-measurement-result storage section is configured so that, for a metabolite on which a predetermined derivatization process is performed before the test sample is analyzed with an analyzer, a quantitative measurement result obtained for a derivatization product formed by the derivatization process is stored and related to the metabolite; and
the display processor is configured to retrieve, from the metabolite-measurement-result storage section, the quantitative measurement result obtained for a derivatization product of a metabolite on the metabolism map, and to insert and display the quantitative measurement result in the showing area for the metabolite on the metabolism map, when the metabolism-map display data stored in the metabolism-map-data storage section is read to display the metabolism map on the display screen.

(Clause 5) In a data processing device for metabolite analysis according to Clause 5, which is one mode of the data processing device for metabolite analysis according to Clause 4, the display processor is configured so that, when quantitative measurement results obtained for a plurality of derivatization products are related to a metabolite and stored in the metabolite-measurement-result storage section, the display processor retrieves, from the metabolite-measurement-result storage section, the quantitative measurement result obtained for a derivatization product that satisfies a predetermined condition among the plurality of derivatization products, and inserts and displays the quantitative measurement result in the showing area for the metabolite on the metabolism map.

(Clause 6) A data processing device for metabolite analysis according to Clause 6 includes:
a metabolite-measurement-result storage section in which, for a metabolite identified based on analysis data acquired by analyzing a test sample with one or more analyzers, quantitative measurement results obtained for a plurality of derivatization products of the metabolite are stored and related to the metabolite;
a metabolism-map-data storage section in which metabolism-map display data for displaying, on a display screen, a metabolism map showing a plurality of metabolites is stored for one or more metabolism maps; and
a display processor configured to retrieve, for each metabolite on the metabolism map, a quantitative measurement result obtained for a derivatization product that satisfies a predetermined condition among the plurality of derivatization products of the metabolite, from the metabolite-measurement-result storage section, and to insert and display the quantitative measurement result in the metabolism map, when the metabolism-map display data stored in the metabolism-map-data storage section is read to display the metabolism map on the display screen.

In the data processing device for metabolite analysis according to Clause 4, 5 or 6, when a metabolite for which a predetermined derivatization process is performed before the test sample is analyzed with the analyzer is contained in the test sample, a quantitative measurement result obtained for a derivatization product formed by the derivatization process can be displayed on the metabolism map as the quantitative measurement result obtained for that metabolite.

In this case, the data processing device for metabolite analysis according to Clause 5 or 6 is configured so that, for a metabolite from which a plurality of derivatization products are formed, a quantitative measurement result obtained for a derivatization product that satisfies a predetermined condition among those derivatization products is on the metabolism map as the quantitative measurement result obtained for the metabolite concerned. The “predetermined condition” may be the derivatization product being superior to the other derivatization products in detection sensitivity, in detection reproducibility, or in stability against heat or chemicals. By using a quantitative measurement result obtained for a derivatization product that satisfies such a condition, a highly reliable metabolism map can be obtained.

### REFERENCE SIGNS LIST

11 . . . GC/MS
111 . . . GC Unit
112 . . . MS Unit
12 . . . LC/MS
121 . . . LC Unit
122 . . . MS Unit
2 . . . Control-and-Processing PC
20 . . . Metabolism-Map-Data Storage Section
21 . . . Metabolite-Information Storage Section
22 . . . Test-Sample-Analysis-Result Storage Section
23 . . . Storage Controller
24 . . . Data Processor
25 . . . Analysis Controller
26 . . . Method Creation Processor
27 . . . Display Processor
28 . . . Information Registration Section
4 . . . Input Unit
41 . . . Metabolite Selector
5 . . . Display Unit
51 . . . Pre-Analysis Display Screen
71 . . . Post-Analysis Display Screen

1. A data processing device for metabolite analysis, comprising:
a metabolite-measurement-result storage section in which a metabolite identified based on analysis data acquired by analyzing a test sample with one or more analyzers and a quantitative measurement result of the metabolite are stored;
a metabolism-map-data storage section in which metabolism-map display data for displaying, on a display screen, a metabolism map is stored for one or more metabolism maps, with each metabolism map showing a plurality of metabolites along with respective quantitative measurement results, and each metabolite having a showing area to which analyzer information representing a type of analyzer capable of detecting the metabolite is to be added; and
a display processor configured to retrieve, for each metabolite on a predetermined metabolism map, a quantitative measurement result corresponding to the metabolite from the metabolite-measurement-result storage section and to display the quantitative measurement result by inserting the quantitative measurement result into the showing area for the metabolite on the metabolism map, when the metabolism-map display data stored in the metabolism-map-data storage section is read to display the predetermined metabolism map on the display screen.

2. The data processing device for metabolite analysis according to claim 1, wherein the one or more analyzers are a gas chromatograph mass spectrometer and a liquid chromatograph mass spectrometer.

3. The data processing device for metabolite analysis according to claim 1, wherein the analyzer information added to the showing area for each metabolite on the metabolism map is color information for displaying at least a portion of the showing area in a color corresponding to the type of analyzer.

4. The data processing device for metabolite analysis according to claim 2, wherein:
the metabolite-measurement-result storage section is configured so that, for a metabolite on which a predetermined derivatization process is performed before the test sample is analyzed with an analyzer, a quantitative measurement result obtained for a derivatization product formed by the derivatization process is stored and related to the metabolite; and
the display processor is configured to retrieve, from the metabolite-measurement-result storage section, the quantitative measurement result obtained for a derivatization product of a metabolite on the metabolism map, and to insert and display the quantitative measurement result in the showing area for the metabolite on the metabolism map, when the metabolism-map display data stored in the metabolism-map-data storage section is read to display the metabolism map on the display screen.

5. The data processing device for metabolite analysis according to claim 4, wherein the display processor is configured so that, when quantitative measurement results obtained for a plurality of derivatization products are related to a metabolite and stored in the metabolite-measurement-result storage section, the display processor retrieves, from the metabolite-measurement-result storage section, the quantitative measurement result obtained for a derivatization product that satisfies a predetermined condition among the plurality of derivatization products, and inserts and displays the quantitative measurement result in the showing area for the metabolite on the metabolism map.

6. A data processing device for metabolite analysis, comprising:
a metabolite-measurement-result storage section in which, for a metabolite identified based on analysis data acquired by analyzing a test sample with one or more analyzers, quantitative measurement results obtained for a plurality of derivatization products of the metabolite are stored and related to the metabolite;
a metabolism-map-data storage section in which metabolism-map display data for displaying, on a display screen, a metabolism map showing a plurality of metabolites is stored for one or more metabolism maps; and
a display processor configured to retrieve, for each metabolite on the metabolism map, a quantitative measurement result obtained for a derivatization product that satisfies a predetermined condition among the plurality of derivatization products of the metabolite, from the metabolite-measurement-result storage section, and to insert and display the quantitative measurement result in the metabolism map, when the metabolism-map display data stored in the metabolism-map-data storage section is read to display the metabolism map on the display screen.
